(19) United States
(12) Patent Application Publication
(10) Pub. No.: US 2018/0117186 A1
(43) Pub. Date: May 3, 2018

(54) MAGNETIC PARTICLES FOR USE IN MAGNETIC RESONANCE IMAGING THERMOMETRY

(71) Applicant: The Regents of the University of Colorado, a body Corporate, Denver, CO (US)

(72) Inventors: Robert Camley, Colorado Springs, CO (US); Zbigniew Celinski, Colorado Springs, CO (US); Janusz H. Hankiewicz, Castle Rock, CO (US)

(21) Appl. No.: 15/801,215

(22) Filed: Nov. 1, 2017

Related U.S. Application Data
(63) Continuation-in-part of application No. PCT/US2016/061866, filed on Nov. 14, 2016.
(60) Provisional application No. 62/415,943, filed on Nov. 1, 2016.

(51) Int. Cl. A61K 49/18; A61B 5/055; A61B 5/01; G01R 33/56; G01R 33/48

(52) U.S. Cl. CPC A61K 49/1824; A61B 5/055; G01R 33/4804; G01R 33/5601; A61B 5/01

(57) ABSTRACT

The present invention provides doped ferrite particles or metallic compounds or alloys, which can be used as temperature-dependent sensors in magnetic resonance imaging. In certain embodiments, these particles have a Curie temperature near that of living animals, allowing one to obtain spatial maps of temperature useful for thermal medical procedures or diagnostics. In other embodiments, one can use the methods and materials of the present invention to obtain spatial temperature maps of materials and non-living objects, such as tires or polymers. This method allows for a non-invasive determination of internal body temperature with a resolution of about 1° C.

### CROSS-REFERENCE TO RELATED APPLICATIONS

This application is a continuation-in-part of PCT Application No. PCT/US2016/061866, having an international filing date of 14 Nov. 2016. This application also claims the benefit of U.S. Provisional Patent Application 62/415,943, filed 1 Nov. 2016. The entireties of both of these applications are incorporated herein by reference in their entireties.

### STATEMENT REGARDING FEDERALLY SPONSORED RESEARCH

This invention was made with government support under Award No. 1651589, titled “I-Corps: Magnetic Resonance Imaging Thermometry Using Ferromagnetic Particles,” awarded by the National Science Foundation to the University of Colorado at Colorado Springs. The government has certain rights in the invention.

### FIELD OF THE INVENTION

This invention relates to magnetic resonance imaging, and specifically to methods of measuring temperatures utilizing a magnetic resonance imaging contrast agent.

### BACKGROUND OF THE INVENTION

Temperature is a fundamental parameter reflecting the biological status of the body and individual tissues. Clinical studies indicate that localized temperature measurements could be a useful method for the detection of a variety of health problems, including certain tumors and inflammations. Precise determination of tissue temperature is also important in various thermal medical intervention procedures. In hyperthermia therapy for selective tumor treatment, the temperature of tumor-affected tissue is raised to 40-43° C. and followed by other cancer treatment modalities. Thermal ablation therapies such as laser, radio-frequency (RF), microwave, and high intensity focused ultrasound therapies utilize much higher temperature exposure (48° C.-100° C.) for tissue necrosis through thermal coagulation. The exact value of applied temperature depends on the type of disease, heating modality, target size and position, and tissue heat conduction and absorption. Additionally, temperature reporting is critical for monitoring the temperature of tissue around medical metallic implants during standard magnetic resonance imaging that is caused by fast switching magnetic gradients and radio-frequency pulses.

Conventional thermometry is usually invasive, allows only single-point temperature measurements, and may interfere with the therapeutic and imaging instrument. The single-point limitation is particularly disadvantageous, because the ability to monitor the temperature of body tissues in vivo in three dimensions is thus important for both diagnosis and treatment of patients.

Thermometry based on the numbers of MR temperature-sensitive tissue parameters, such as shift of proton resonance frequency (PRF), diffusion coefficient, T₁ and T₂ nuclear relaxation times, magnetization transfer, and proton density, has been developed. For its high accuracy (0.2° C. in phantoms), linearity, and wide temperature range, PRF is the current gold standard for temperature measurements in aqueous tissues. However, as these methods rely on comparison to a baseline image, they are sensitive to motion and thermally-induced susceptibility artifacts during scanning, which prevent highly accurate in vivo MRI thermometry.

Some of the limitations of conventional thermometry can be addressed by MRI thermometry techniques that produce temperature maps having advantageous thermal, spatial, and temporal resolution, which may be superimposed on anatomical images within the targeted tissue. MRI thermometry is an emerging field that allows for non-invasive and in vivo measurements of temperature deep inside a body or a mass of material. This is accomplished by introducing into the body or mass a magnetic contrast agent, the magnetization of which changes rapidly with temperature. The requirement for a rapid change in magnetization as a function of temperature can be met by using any magnetic material near its Curie transition temperature (T_{C}). Dipolar fields produced by particles of the magnetic contrast agent embedded in tissue or other materials change the local homogeneity of the static magnetic field of the MRI scanner. This broadens the nuclear magnetic resonance (NMR) linewidth and consequently, depending on temperature, darkens T₂* weighted images. As a result, the NMR linewidth or the brightness of the MR images can be calibrated to obtain a local value of temperature or to produce 3D maps of an entire region of interest.

The practical importance of MRI temperature contrast for obtaining 3D temperature maps is very important. Most applications are in the biomedical area, for the detection of local inflammations and assisting in different heating procedures such as hyperthermia and thermal ablation in tumor treatment. However, MRI thermometry may also be applicable in material science and in industries where, by way of non-limiting example, non-invasive determination of temperature maps of an interior of a sample exposed to thermal stress may be essential for failure analysis.

There is thus a need in the art for nontoxic materials which can produce significant changes in MRI intensity as a function of temperature.

### SUMMARY OF THE INVENTION

The present invention provides an MRI thermometry contrast agent, comprising a ferrite compound of the formula (M₁₋ₓYₓ)_{z}Fe_{3-z}O₄, wherein M and Y represent metallic elements, wherein the ferrite compound has a Curie transition temperature between about 150 K and about 350 K, and wherein a magnitude of a change in magnetization of the ferrite compound as a function of temperature, in a magnetic field of about between about 0.2 teslas and about 7 teslas and a temperature range between about 278 K and about 333 K, is at least about 0.05 Am²·kg⁻¹·K⁻¹.

In embodiments, the ferrite compound may be in the form of particles. A mean particle size of the particles may be between about 5 nm and about 10 μm.

The present invention also provides a pharmaceutical composition suitable for administration to a human subject, comprising a ferrite compound of the formula: (M₁₋ₓYₓ)_{z}Fe_{3-z}O₄, wherein M and Y represent metallic elements, wherein the ferrite compound has a Curie transition temperature between about 150 K and about 350 K, and wherein a magnitude of a change in magnetization of the ferrite compound as a function of temperature, in a magnetic field of about 3.0 teslas and a temperature range between about 278 K and about 333 K, is at least about 0.01 Am²·kg⁻¹·K⁻¹; and at least one pharmaceutically acceptable carrier or excipient.

In embodiments, the composition may be suitable for administration to a human subject by a route selected from the group consisting of inhalational, oral, nasal, rectal, parenteral, sublingual, transdermal, transmucosal, intravesical, intrapulmonary, intraduodenal, intragastrical, intrathecal, epidural, intrapleural, intraperitoneal, intratracheal, otic, intraocular, subcutaneous, intramuscular, intradermal, intra-arterial, intravenous, intrabronchial, and topical.

In embodiments, a concentration of the ferrite compound in the composition may be between about 0.04 mM and about 4 mM.

The present invention further provides a method for non-invasively measuring a temperature of a human subject, comprising administering to the subject a composition comprising a ferrite compound of the formula (M₁₋ₓYₓ)_{z}Fe_{3-z}O₄, wherein M and Y represent metallic elements, wherein the ferrite compound has a Curie transition temperature between about 150 K and about 350 K, and wherein a magnitude of a change in magnetization of the ferrite compound as a function of temperature, in a magnetic field of about 3.0 teslas and a temperature range between about 278 K and about 333 K, is at least about 0.01 Am²·kg⁻¹·K⁻¹; collecting at least one magnetic resonance image of the subject; measuring at least one relaxation time of the composition in the subject, the at least one relaxation time selected from the group consisting of T₁ relaxation time, T₂ relaxation time, and T₂* relaxation time; and determining the temperature of the human subject using the at least one magnetic resonance image and the at least one relaxation time.

In embodiments, a measurement error of the measured temperature of the subject may be no more than about 2° C., and preferably no more than about 1° C.

The present invention further provides an article for use in MRI thermometry of a human subject, comprising a medium, comprising a nuclide with non-zero nuclear spin; and a ferrite compound of the formula (M₁₋ₓYₓ)_{z}Fe_{3-z}O₄, wherein M and Y represent metallic elements, wherein the ferrite compound has a Curie transition temperature between about 150 K and about 350 K, and wherein a magnitude of a change in magnetization of the ferrite compound as a function of temperature, in a magnetic field of about 3.0 teslas and a temperature range between about 278 K and about 333 K, is at least about 0.01 Am²·kg⁻¹·K⁻¹, wherein a concentration of the ferrite compound in the composition is between about 0.05 g/L and about 5 g/L.

In embodiments, the article may be selected from the group consisting of a phantom in the form of a container or in the form of a thick (preferably polymeric) film, and an outer layer of a device. Exemplary devices include a catheter, an MRI-guided treatment tool, an implant, a probe, an applicator, a mesh, and a stent. Where the article is a phantom in the form of a container, the container may be suitable for placement in a body cavity of the subject and may be selected from the group consisting of an ampoule and a film. Where the article is a phantom in the form of a film, the film may be configured to cover skin of the subject and allow MRI thermometry of the skin during an MRI-guided medical procedure.

The invention further provides a method for non-invasively measuring an internal temperature of an object, the object comprising a nuclide with non-zero nuclear spin, the method comprising placing in an interior of the object a composition comprising a ferrite compound of the formula (M₁₋ₓYₓ)_{z}Fe_{3-z}O₄, wherein M and Y represent metallic elements, wherein the ferrite compound has a Curie transition temperature between about 150 K and about 350 K, and wherein a magnitude of a change in magnetization of the ferrite compound as a function of temperature, in a magnetic field of about 3.0 teslas and a temperature range between about 278 K and about 333 K, is at least about 0.01 Am²·kg⁻¹·K⁻¹; collecting at least one magnetic resonance image of the object; measuring at least one relaxation time of the composition in the object, the at least one relaxation time selected from the group consisting of T₁ relaxation time, T₂ relaxation time, and T₂* relaxation time; and determining the temperature of the object using the at least one magnetic resonance image and the at least one relaxation time.

Any of several parameters of an MRI contrast agent of the present invention, including, by way of non-limiting example, particle size, chemical composition, and concentration, may be adjusted or controlled to make the contrast agent especially suitable for a particular application, as will be apparent to those of ordinary skill in the art based on the below Detailed Description. By way of non-limiting example, the chemical composition of the contrast agent may be modified to make the contrast agent effective over a desired range of temperatures, and the particle size of the contrast agent may be modified to allow for temperature determination by measurement of a desired relaxation time.

### BRIEF DESCRIPTION OF THE DRAWINGS

FIG. 1 shows the temperature dependence of magnetization in gadolinium powder for selected magnetic fields in a superconducting quantum interference device;

FIG. 2 shows the temperature changes of relative NMR line broadening in a mixture of 1% agar gel with suspended gadolinium powder with an applied magnetic field of 364 mT;

FIG. 3 shows an example of gradient-echo MRI images of phantoms at different temperatures;

FIG. 4 shows the thermal changes of ratios of images intensity of pure agar to agar with gadolinium particles for various gadolinium concentrations;

FIG. 5 shows an example MRI image of pure agar gel and agar gel with gadolinium particles at 10° C. and 45° C.;

FIG. 6 shows the temperature dependence of the magnetic moment in gadolinium powder for selected magnetic fields;

FIG. 7 shows the temperature changes of local magnetic field inhomogeneity in a mixture of 1% agar gel and gadolinium powder;

FIGS. 8A and 8B both show MRI gradient echo images of pure agar gel (top) and agar gel with gadolinium particles (bottom, left and right) at 10° C. (FIG. 8A) and 45° C. (FIG. 8B);

FIG. 9A is a set of graphs reporting mass magnetization measurements versus temperature of Cu_{0.35}Zn_{0.65}Fe₂O₄ ferrite at different magnetic fields;

FIG. 9B shows magnified results of FIG. 9A at a low magnetic field of 2 mT used for T_{C} temperature determination;

FIG. 10A is a graph showing the temperature dependence of low-field (364 mT) measurements of T₂ nuclear spin-spin relaxation time in agar with embedded Cu_{0.35}Zn_{0.65}Fe₂O₄ particles;

FIG. 10B is a graph showing the temperature dependence of low-field (364 mT) measurements of the difference between the temperature-dependent linewidths for the agar sample with and without the ferrite particles;

FIG. 11 is a set of images showing T₂* weighted, gradient-echo MR images in a 2% Ringer's solution-agarose sample with embedded Cu_{0.35}Zn_{0.65}Fe₂O₄ particles at different temperatures;

FIG. 12 reports temperature changes of relative MR image intensity in ferrite doped agar as analyzed using images from FIG. 11;

FIG. 13 is a set of images showing T₂* weighted, gradient-echo MR images in a 2% Ringer's solution-agarose sample with embedded Co_{0.3}Zn_{0.7}Fe₂O₄ nanoparticles at different temperatures; and

FIG. 14 shows the nuclear spin-lattice relaxation times T₁ of water protons as a function of temperature in pure agar gel and agar gel with various Co_{0.3}Zn_{0.7}Fe₂O₄ nanoparticle concentrations at 364 mT.

### DETAILED DESCRIPTION OF THE INVENTION

As used herein, each of the following terms has the meaning associated with it in this section. Unless defined otherwise, all technical and scientific terms used herein generally have the same meaning as commonly understood by one of ordinary skill in the art to which this invention belongs. Generally, the nomenclature used herein and the laboratory procedures in materials chemistry and physics, medical imaging, magnetic measurements and pharmaceutical science are those well-known and commonly employed in the art.

As used herein, the articles “a” and “an” refer to one or to more than one (i.e. to at least one) of the grammatical object of the article. By way of example, “an element” means one element or more than one element.

As used herein, the term “about” is understood by persons of ordinary skill in the art, and its meaning depends, to some extent, on the context in which it is used. As used herein when referring to a measurable value such as an amount, a temporal duration, and the like, the term “about” is meant to encompass variations of ±20% or ±10%, more preferably ±5%, even more preferably ±1%, and still more preferably ±0.1% from the specified value, as such variations are appropriate to perform the disclosed methods.

As used herein, the term “pharmaceutically acceptable carrier” refers to a pharmaceutically acceptable material, composition, or carrier, such as a liquid or solid filler, stabilizer, dispersing agent, suspending agent, diluent, excipient, thickening agent, solvent, or encapsulating material, involved in carrying or transporting a compound useful within the invention within or to the subject such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the subject, i.e. biocompatible. Some examples of materials that may serve as pharmaceutically acceptable carriers include: sugars, such as lactose, glucose, and sucrose; starches, such as corn starch and potato starch; cellulose and its derivatives, such as sodium carboxymethyl cellulose, ethyl cellulose, and cellulose acetate; powdered tragacanth; malt; gelatin; talc; excipients, such as cocoa butter and suppository waxes; oils, such as peanut oil, cottonseed oil, safflower oil, sesame oil, olive oil, corn oil, and soybean oil; glycols, such as propylene glycol; polyols, such as glycerin, sorbitol, mannitol, and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, the term “pharmaceutically acceptable carrier” also refers to any coating, antibacterial or antifungal agent, absorption delaying agent, and the like that is compatible with the activity of the compound useful within the invention, and is physiologically acceptable to the subject. Supplementary active compounds may also be incorporated into the compositions. The “pharmaceutically acceptable carrier” may further include a pharmaceutically acceptable salt of the compound useful within the invention. Other additional ingredients that may be included in the pharmaceutical compositions used in the practice of the invention are known in the art and described, for example, in Remington's Pharmaceutical Sciences (Genaro, Ed., Mack Publishing Co., 1985, Easton, Pa.), which is incorporated herein by reference.

As used herein, the term “pharmaceutical composition” refers to a mixture of at least one compound useful within the invention with a pharmaceutically acceptable carrier. The pharmaceutical composition facilitates administration of the compound to a subject.

As used herein, the term “subject” refers to a human or non-human mammal or a bird. Non-human mammals that may be “subjects” as that term is used herein include, by way of non-limiting example, livestock and pets, such as ovine, bovine, porcine, canine, feline, and murine mammals. In some embodiments, a “subject” as that term is used herein may be human.

### Gadolinium-Based Temperature-Sensitive MRI Contrast Agents

The present disclosure provides non-invasive methods of temperature measurement within tissue that may utilize temperature changes of the net magnetic moment of magnetic particles. The magnetic particles embedded in or near the tissue create a local dipole magnetic field that modulate the homogeneity of the main static magnetic field of the MRI scanner and broaden the NMR line. Consequently, the effective nuclear spin-spin relaxation time (T₂*) of the tissue near the magnetic particles will be shortened. This effect may then be measured directly with image-guided localized NMR spectroscopy as linewidth broadening. The linewidth broadening can be visible as a darker area on MRI images acquired with the gradient echo method, which is very sensitive for local field inhomogeneity. Different line widths, or shades of gray, may be calibrated to obtain a map of temperature or to report the achievement of a certain temperature threshold in a specific tissue during interventional procedures.

Various methods may be utilized to adjust the transition temperature of the magnetic particles that form a contrast agent used in these methods of temperature measurement. For example, smaller or larger magnetic particles may be used. The magnetic particle size may be varied, by way of non-limiting example, between about 5 nm and about 10 μm. Generally, using smaller magnetic particles moves the transition temperature down. While the use of smaller particles is not a useful method for gadolinium, the change in T_{c} based on particle size may be useful for other materials. Additionally, different alloy compositions may be utilized. For example, Permalloy Ni₈₀Fe₂₀ has a T_{c} of 576° C. With copper doping (48.5%), however, the T_{c} may be reduced to 43° C. Further, dopants may be added which have a higher exchange coupling. For example, Co—Gd exchange is four times stronger than Gd—Gd exchange. The Co—Gd coupling stabilizes the gadolinium against thermal fluctuations, but cobalt couples antiparallel to gadolinium, reducing the net moment.

Gadolinium possesses a relatively high magnetic moment and, in zero applied field, is characterized by a transition from a ferromagnetic state to a paramagnetic state around 273 K (about 0° C.). The gradient echo images of phantoms at 1.5 T with various concentrations of gadolinium particles in agar gel have shown significant changes in image intensity as a function of temperature. A gadolinium concentration of only 0.69 mM allowed for a temperature determination with a resolution of less than 1° C. in the temperature range between 284 K and 310 K (between about 11° C. and about 37° C.). However, gadolinium is toxic, and any use in vivo would require a secure outer shell, membrane, or other layer that prevents the gadolinium from contacting body tissues. Thus, it is important to look for other bio-compatible materials.

### Ferrite-Based Temperature-Sensitive MRI Contrast Agents

In contrast to gadolinium, iron oxide ferrites are nontoxic and have been approved by the Food and Drug Administration (FDA) for use in humans. Accordingly, the present invention can also utilize doped Fe₃O₄ ferrites for MRI thermometry, and the present inventors have demonstrated that MgZn, CoZn, and CuZn ferrites can be used to obtain spatial maps of temperature by MRI. A general chemical formula for ferrite materials suitable for use in the present invention is (M₁₋ₓZnₓ)_{z}Fe_{3-z}O₄, where M is magnesium (Mg), cobalt (Co), or copper (Cu), but other divalent and trivalent metals, e.g. calcium, barium, platinum, etc., can be used as dopants to maintain the Curie temperature T_{c} of the ferrite material at or near body temperature or another temperature of interest.

The present invention demonstrates the utility of ferrite compounds having the general chemical formula (M₁₋ₓYₓ)_{z}Fe_{3-z}O₄, where M and Y represent metallic elements, in obtaining spatial maps of temperature via MRI thermometry. In certain embodiments, ferrite compounds having chemical formulas of Cu_{0.35}Zn_{0.65}Fe₂O₄, (Mg_{0.32}Zn_{0.68})_{1.05}Fe_{1.95}O₄, Mg_{0.4}Zn_{0.6}Fe₂O₄, and/or Co_{0.3}Zn_{0.7}Fe₂O₄ may be used. Those of ordinary skill in the art will understand how to adjust the chemical composition of the ferrite material for use in a particular application, depending on, by way of non-limiting example, the magnetic field used by a particular clinical MRI scanner, e.g. about 1.5 T, about 3 T, or about 7 T. In some embodiments, the ferrite material(s) may be in the form of nanoparticles. In other embodiments, the ferrite material(s) may be in the form of particles having diameters of between about 0.5 micrometers and about 5 micrometers.

In certain embodiments, the invention provides a suspension, wherein the ferrite particles are suspended in a liquid, a mixture or solution of liquids, or a gel. In other embodiments, the suspension medium in which the ferrite particles are suspended can be selected from the group consisting of Ringer's solution, agar gel, and any other isotonic solution similar to mammalian body fluids. In other embodiments, the compositions of the invention can be formulated with a pharmaceutically acceptable organic or inorganic carrier substance or medium suitable for administration to a living human subject. In other embodiments, the composition is formulated to be suitable for administration by a method selected from the group consisting of inhalational, oral, nasal, rectal, parenteral, sublingual, transdermal, transmucosal (e.g. lingual or sublingual, buccal or transbuccal, urethral or transurethral, transvaginal or perivaginal, nasal or intranasal, and rectal or transrectal), intravesical, intrapulmonary, intraduodenal, intragastrical, intrathecal, epidural, intrapleural, intraperitoneal, intratracheal, otic, intraocular, subcutaneous, intramuscular, intradermal, intra-arterial, intravenous, intrabronchial, and topical administration. In other embodiments, the concentration of the ferrite compound in the at least one pharmaceutically acceptable carrier is between about 0.1 mM and about 0.4 mM.

In certain embodiments, magnetic particles may be distributed through a hydrogen-rich medium forming a phantom in a container, e.g. in an ampoule or a film, that can be placed in a body cavity. In certain embodiments, magnetic particles may be distributed in the walls of catheters or in protective sheathings of instruments used during MRI-guided ablation. In other embodiments, magnetic particles may be distributed in a proton-rich medium (phantoms in the form of a cap or thick film) that can cover the skin of the patient and allow measurement of the temperature near the skin during thermal procedures, e.g. measurement of the temperature of the skull during an MRI-guided focused ultrasound ablation procedure. One advantage of this approach over the approaches of the prior art is that it allows retracting the phantom (ampule, catheters, cap, etc.) during or after the procedure without exposing tissues to the magnetic particles, which reduces risk to the patient and can simplify FDA approval.

In certain embodiments, magnetic particles can be distributed in a part of a non-metallic body-safe material, e.g. a covering of an implant, a probe, an applicator, a mesh, or a stent, such that MRI can be used to monitor temperature during or after a medical procedure. In these embodiments, magnetic particles are not introduced into the blood stream or tissue of the patient at all.

Ferrite particles suitable for use in the present invention can be prepared by many methods. One such method comprises solid-state sintering at temperatures between about 800° C. and about 1200° C., followed by grinding. Another method comprises hydrolysis of metallic salts in an aqueous solution. Other possible methods that may be used to prepare appropriate ferrite particles with the desired properties include, by way of non-limiting example, sol gel techniques.

Ferrite particles for use in medical applications of some embodiments of the present invention may exhibit a strong temperature dependence of magnetization near human body temperature. The Curie temperatures of such ferrite materials can be modified by adjusting the chemical composition of the material. In certain embodiments, MRI may not measure the change of magnetization directly but rather the change in relaxation times T₁, T₂, and T₂* due to the change in magnetization. Any or all of these measurements may be used to determine the temperature of a body or a mass of material.

The present invention is also suitable for providing temperature maps in composite materials, including, by way of non-limiting example, tires. In many applications, including tires, ZnO may be added to a composite material to obtain certain desired material properties. A small addition of magnetic oxide (ferrite) particles according to the present invention does not noticeably change the mechanical properties of the material, but allows the temperature of the material to be measured by MRI. In some embodiments, ferrite particles can be introduced during the fabrication process to allow for the visualization of temperature at any time during fabrication, for example to allow for a better understanding of a vulcanization process. The magnetic particles of the present intention may be designed to exhibit a very strong temperature-dependent magnetization, and as a result such particles, distributed in a composite matrix, will affect the precessional frequency of protons or other nuclei, such as ¹³C or ¹⁹F. As a result, an NMR linewidth may broaden, and such broadening can be measured, for example, by T₂* weighted MRI imaging.

In addition to studies of living objects, the invention further provides methods of non-invasively determining a spatial map of temperature in general condensed matter objects by MRI. Such methods may use T₂* weighted images and/or T₁ and/or T₂ images. In addition to the change in magnetization, concentrations of the ferrite material may be varied to obtain optimum images.

The invention further provides a method for non-invasively measuring the internal temperature of a subject, the method comprising: administering a composition comprising a magnetic compound to the subject; collecting a magnetic resonance image of the subject; and determining the localized internal temperature of an area of the subject's body using the intensity of the MR signal of the magnetic compound. For use in the human body, the compound may be a doped ferrite with a general chemical formula of (M₁₋ₓYₓ)_{z}Fe_{3-z}O₄, where M and Y represent metallic elements and x and z are chosen to obtain a desired Curie temperature, T_{C}, of the material. The Curie temperature of the material may typically, but need not, be equal to or near human body temperature. For studies of materials, the magnetic particles can also include copper-doped Permalloy, gadolinium, or other metallic elements, which may be chosen to provide an appropriate Curie temperature for the particular application.

In certain embodiments, the compounds and compositions of the invention can be administered to a subject as part of a pharmaceutical formulation. In certain embodiments, the method can determine the localized internal temperature of a subject with a resolution of less than 2° C.

The disclosure now being generally described will be more readily understood by reference to the following examples, which are included merely for the purposes of illustration of certain aspects of the embodiments of the present disclosure. The examples are not intended to limit the disclosure, as one of skill in the art would recognize from the above teachings and the following examples that other techniques and methods can satisfy the claims and can be employed without departing from the scope of the claimed disclosure.

### EXAMPLES

### Example 1

### Preparation and Performance of 5 μm Gadolinium Particles

In this example, gadolinium particles were prepared by a mechanical method that resulted in the particles having an average grain size of 5 μm. The temperature effect of the gadolinium particles on the ¹H NMR line broadening and MRI image intensity was determined using gadolinium particles suspended in a 1% agar-Ringer's solution gel. This created an isotonic solution similar to the bodily fluids of an animal and prevents the sedimentation of the particles. For NMR and MRI measurements, a mixture containing 20 cc of 1% agar-Ringer's solution and 10 mg of gadolinium powder was prepared (100% concentration). The sample was diluted with 1% agar-Ringer's solution to obtain additional mixtures of 50%, 25%, and 12.5% of the maximum concentration. The mixtures were kept in a liquid state at 90° C. in a water bath and constantly stirred before transferring to 5 mm tubes for 0.12 cc NMR studies, and to Nalgene cryogenic plastic vials to make an MRI phantom. Mixtures were rapidly cooled in ice water to preserve an even distribution of gadolinium particles in the gel.

The magnetization of the gadolinium powder was measured in the range of 0° C. to 60° C. at different magnetic fields using a superconducting quantum interference device (SQUID) magnetometer to determine the temperature dependence of the magnetization and Curie point.

To obtain temperature dependence of the NMR line width, a low field (364 mT/15.5 MHz) pulsed spectrometer was used. The application of the low magnetic field allowed for a minimized shift of T_{C} toward higher temperatures. During the NMR measurements, the samples were cooled and ¹H NMR spectra were taken after the temperature was stabilized, from 5° C. to 50° C., at 5° C. increments.

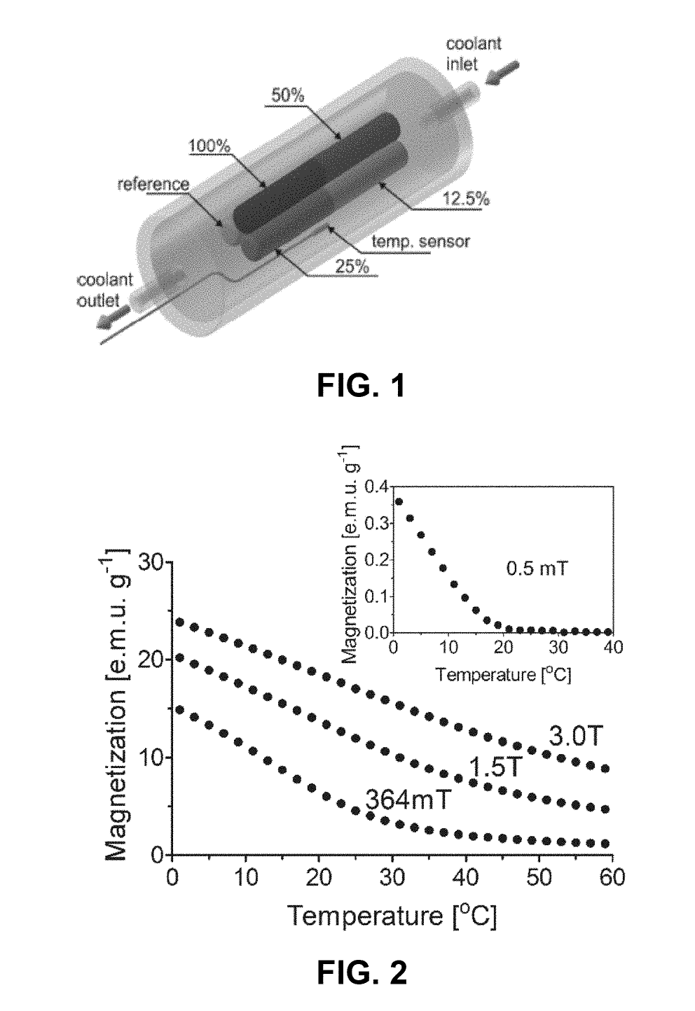

Temperature-dependent MRI images of phantom containing gadolinium particles in agar gel were taken using a preclinical scanner with a 1.5 T, 30 cm bore magnet equipped with a temperature control system. A schematic diagram of the temperature setup is shown in FIG. 1. The phantom consisted of three Nalgene plastic vials (10 mm inner diameter and 80 mm long) placed inside a polycarbonate cell. One vial contained pure agar-Ringer's solution (two concentrations in one vial). The continuous flow of proton-less perfluorocarbon (fluorinert) coolant through the cell forced by a standard circulating bath stabilized phantom temperature without contaminating ¹H images with additional signals. Multi-slice imaging having a FOV of 3×3 cm, a slice thickness of 3 mm, a 64×64 matrix, and a TE of 2.5 ms was used. A long repetition time of 5.0 s was used to avoid signal loss due to relatively long T₁ relaxation time. The phantom temperature was monitored by signal conditioner using a high-precision fiberoptic sensor placed in the space near vials.

FIG. 2 shows the results of SQUID measurements at selected magnetic fields. A low magnitude field of 0.5 mT was used to determine the transition of the gadolinium powder from the ferromagnetic state to the paramagnetic state. The Curie temperature of the sample was estimated as approximately 19° C. Larger values of magnetic field were applied to match fields of NMR spectrometer (364 mT) used for measurement of NMR line broadening and commercially available clinical MRI scanners (1.5 T, 3.0 T, and 7 T). FIG. 2 demonstrates that an increase of magnetic field shifts the transition to the paramagnetic state toward higher temperatures and makes regular gadolinium particles a useful temperature-sensitive contrast agent within a temperature range suitable for MRIs of human subjects.

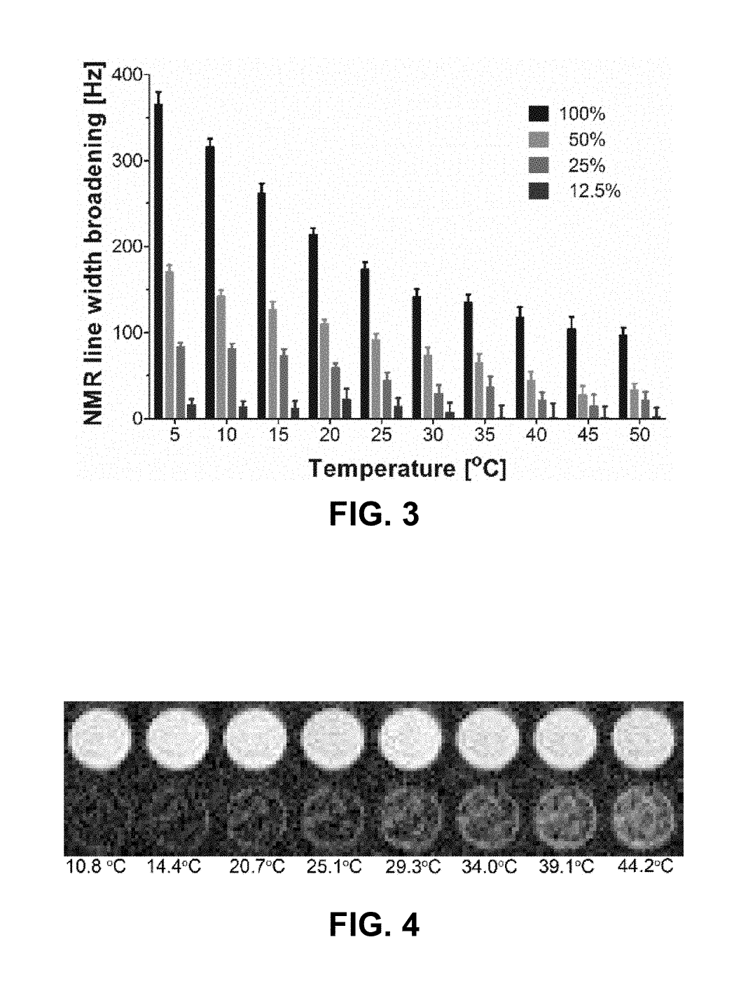

FIG. 3 shows the thermal dependence of the NMR linewidth broadening due to the presence of gadolinium particles. A Fourier transform of free induction signal from the water was used to determine the linewidth. The results of relative line broadening were obtained by subtracting the NMR linewidth at full width at half maximum (FWHM) in pure agar gel from the linewidth at FWHM in agar gel with suspended gadolinium particles.

FIG. 4 shows images of the sample with the maximum gadolinium concentration and the sample with pure agar. The top row is undoped agar gel and the bottom row shows the agar gel doped with the highest content of gadolinium particles (100%). The images of agar gel with gadolinium show a strong temperature-dependent increase in brightness.

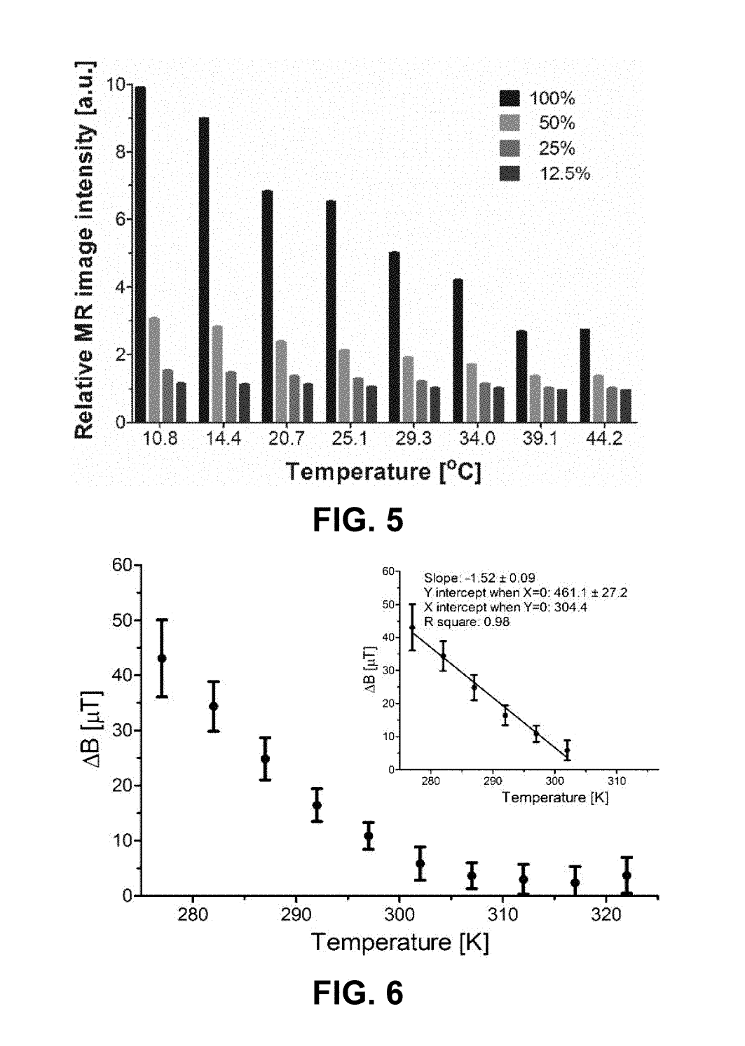

FIG. 5 shows temperature-dependent relative MR intensity (ratio of image intensity of pure agar gel to image intensity of gel with gadolinium particles) for various gadolinium concentrations. The image intensity was calculated across the entire axial slice using a MATLAB platform program. These data demonstrate that several different concentrations of gadolinium allow for temperature-dependent measurements.

Analysis of SQUID and NMR data demonstrates a strong correlation (p<0.001) between the magnetic moment and NMR linewidth broadening for 100% gadolinium concentration. Linear parts of line width broadening (temperature range 5° C. to 30° C. in FIG. 3) and ratios of MR image intensity (temperature range 10.8° C. to 39.1° C. in FIGS. 4 and 5) for 100% concentration of gadolinium were statistically analyzed using regression of means. Results demonstrate that both line width broadening and image intensity ratios are strongly correlated (R²=0.99) with temperature changes. From the regression's 95% confidence bands, the accuracy of temperature determination in the phantom is ±1.0° C. at 16° C. using NMR linewidth broadening, and ±1.2° C. at 24° C. using MR image intensity.

These results demonstrate that the NMR linewidth of ¹H is strongly affected by the presence of gadolinium particles in aqueous solutions. Gradient echo images of phantoms at 1.5 T with various concentrations of gadolinium particles show strong intensity increase when temperature is changed from about 10.8° C. to about 39.1° C. (FIGS. 4 and 5), allowing for temperature determination with an accuracy of ±1.2° C. at 24° C. This demonstrates that gadolinium is a promising element in designing an MRI temperature contrast agent and that magnetic particles with substantial thermal changes of magnetization are suitable for temperature measurements as temperature-sensitive MRI contrast agents.

### Example 2

### Preparation and Performance of 10 μm Gadolinium Particles

In this example, small gadolinium particles having an average size of 10 μm were used. Among the different ferromagnetic metals, gadolinium is characterized by transition from a ferromagnetic state to a paramagnetic state at temperatures of about 20° C., close to human body temperature. Gadolinium also possesses a large magnetic moment, allowing it to create a local dipolar field, the magnitude of which depends strongly on temperature.

The magnetic properties of gadolinium powder were measured in the temperature range from 272 K to 334 K at different magnetic fields using SQUID. The temperature effect of the presence of gadolinium on ¹H NMR line broadening was then determined using gadolinium particles suspended in 1% agar-deionized water gel. To lower the effect of the magnetic field on the shift of the Curie point, a pulsed NMR spectrometer operating at a low magnetic field (364 mT/15.5 MHz) was used. Finally, six different concentrations of gadolinium particles were used to be phantom tested using a 1.5 T MR imager at two temperatures, 10° C. and 45° C.

FIG. 6 shows the SQUID results at selected magnetic fields. A low magnitude field of 2.5 mT was used to determine the transition of the gadolinium powder from the ferromagnetic state to the paramagnetic state. The Curie temperature of the sample was estimated to be approximately 292 K (about 19° C.). Larger values of magnetic field were applied to match fields of NMR spectrometer (364 mT) used for measurement of NMR line broadening and of commercially available clinical MRI scanners (1.5 T and 3.0 T). FIG. 1 demonstrates that an increase of magnetic field shifts the transition to paramagnetic state toward higher temperatures and makes regular gadolinium particles a useful temperature sensitive contrast within the target temperature range.

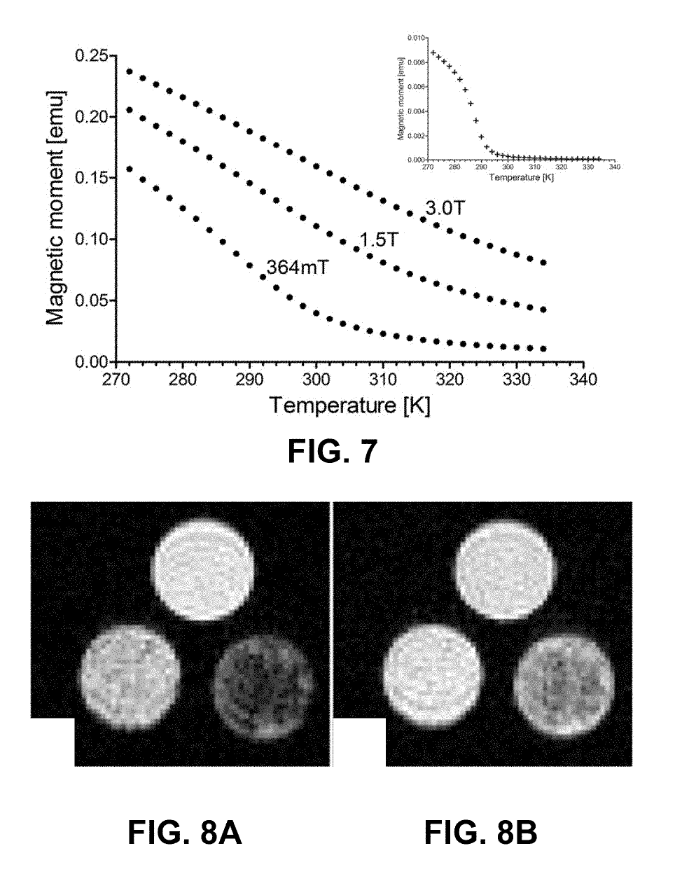

FIG. 7 shows the thermal dependence of the magnetic field inhomogeneity due to the presence of gadolinium particles. A mixture containing 20 mL of 1% agar gel and 10 mg of gadolinium powder was prepared, kept in a liquid state at 90° C. in a water bath, and constantly stirred. To make the NMR sample, a small amount (about 0.12 mL) of hot, liquid mixture was transferred to a standard 5 mm NMR glass tube and rapidly cooled to preserve an even distribution of gadolinium particles in the gel. During NMR measurements, the sample was further cooled, and ¹H spectra were taken after the temperature was stabilized at 5 K increments from 278 K to 323 K. The Fourier transform was used to determine the line broadening. The results were obtained by subtracting the NMR linewidth at FWHM in pure agar gel from linewidth at FWHM in agar gel with suspended gadolinium particles. Then line broadening (in Hz) was converted to a magnetic field inhomogeneity (in μT).

Gradient echo images of cylindrical phantoms made of different concentrations of gadolinium particles in 1% Ringer's solution-agar gel were taken at 10° C. and 45° C. FIG. 8 shows selected images of two different concentrations of gadolinium and a control sample of pure agar gel.

These results demonstrate that the NMR line width of ¹H is strongly affected by the presence of gadolinium particles and changes due to the thermal changes of the particles' magnetic moment and can be used as a temperature sensitive parameter for temperature measurements. Regression analysis (FIG. 7) shows a strong linear dependence (R²=0.98) of line broadening in a range of 278 K to 303 K with a strong slope of 1.5 μT/K or 64 Hz/K. Temperature-stimulated change in NMR linewidth in the phantom is correlated with MR image intensity changes (FIG. 8). As the magnetic moment of particles drops as temperature is increased, MR images get significantly brighter.

### Example 3

### Synthesis of Ferrite Compositions and Ferrite Doping Optimization

The temperature dependence of the magnetization of ferrite materials of the present invention may be calculated by a simple theoretical model. Within a mean field theory, the thermal averaged magnitude of a spin, S, is given by:

<S>=SBs(x)

where Bs is the Brillouin function and x is the ratio of the magnetic energy to the thermal energy given by:

$$x = \frac{g {μ}_{B} S ( H + λ 〈 S 〉 )}{kT}$$

Here H is the applied field; g is the Landé g-factor; μ_{B} is the Bohr magneton; k is Boltzmann's constant; T is temperature; and λ<S> measures the exchange field produced on a given spin. The exchange constant λ is found from the experimental Curie temperature.

Several compositions of Cu₁₋ₓZnₓFe₂O₄ ferrites were made using a standard solid-state sintering method at 1403 K, as will be well-known and understood by those of ordinary skill in the art. X-ray diffraction patterns show a pure spinel phase. The magnetic properties of the ferrites were studied using a SQUID magnetometer to find the best composition, one with a large magnetic moment that decreases rapidly near body temperature. From the different compositions, the present inventors chose Cu_{0.35}Zn_{0.65}Fe₂O₄ as a preferred embodiment because it gives the fastest reduction in magnetization with temperature in the range of 278 K to 333 K.

To produce the particles, sintered pellets of ferrite were ground to a powder in a mortar and sieved. The mean diameter of the particles was determined by scanning electron microscopy to be 3.8 μm; this sieved powder was used in the magnetization, Mössbauer, NMR, and MRI experiments. For the NMR studies, performed at 364 mT, the ferrite particles were suspended in a 1% Ringer's solution-agar gel with a concentration of 3.5 mM to determine the thermal changes of resonance linewidth (full width at half maximum, FWHM) of water protons. For the MRI temperature measurements, a phantom consisting of two cylindrical objects (10 mm in diameter and 30 mm long) was used: one volume contained pure 2% Ringer's solution-agar gel, serving as a reference, and the second volume contained the identical gel with embedded ferrite particles with a concentration of 1 mM. The internal temperature of the phantom was stabilized at multiple points by the flow of a fluorocarbon-based, non-protonic fluid as described above in Example 1. The gradient echo method (GEM), known for its inherent sensitivity to local magnetic field inhomogeneity, was used for MR imaging with the following parameters: axial slice orientation, field of view 30×30 mm², in-plane resolution 0.47 mm/pixel, slice thickness 4 mm, repetition time 100 ms, echo time 2.5 ms, radio-frequency flip angle 20°. The imaging was conducted in a preclinical MR scanner with a 30 cm bore and at a magnetic field of 3 T.

### Example 4

### Magnetization Measurements

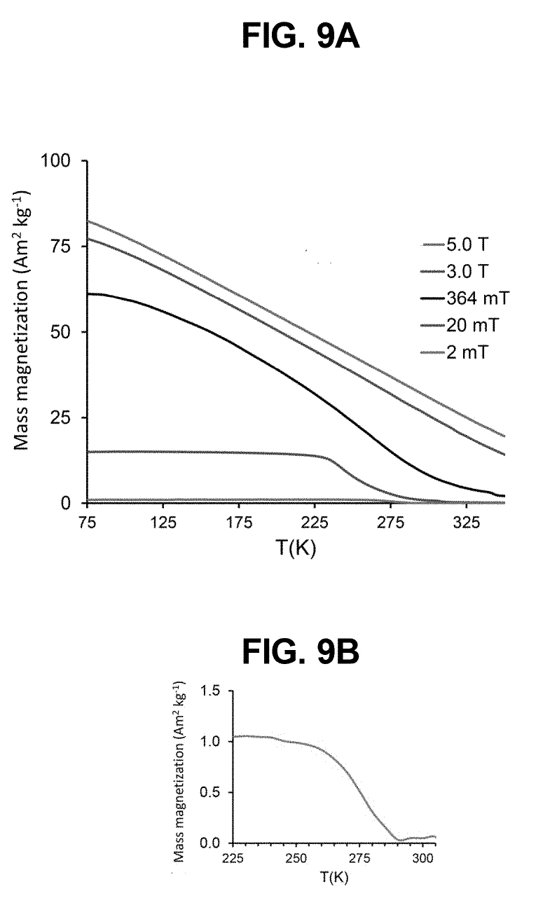

A solid 2.1 mg sample was initially field-cooled to 5 K in a magnetic field of 5 T in a SQUID magnetometer. The magnetic field was then reduced to the desired value at the start of the measurements. FIG. 9A summarizes the magnetization results. In the strongest applied magnetic fields of 3 T and 5 T, a strong and nearly linear decrease of the magnetization was observed as temperature increased. At 3.0 T in the temperature range of 278 K to 333 K, the slope is −0.238±0.002 Am²·kg⁻¹·K⁻¹. This value is comparable to a slope of −0.245±0.001 Am²·kg⁻¹·K⁻¹ found for the 5 μm gadolinium particles used in previous studies at the same magnetic field. However, at a temperature of 310 K, i.e. approximately human body temperature, the mass magnetization in the Cu_{0.35}Zn_{0.65}Fe₂O₄ ferrite is almost twice as high as in gadolinium; 23.1 Am²·kg⁻¹ and 13.0 Am²·kg⁻¹, respectively. This suggests that a smaller amount of contrast material could be used to achieve similar temperature measurement accuracy when the ferrite is compared to gadolinium. The experiments in a 2 mT field allowed a precise determination of the Curie temperature (290 K) as seen in FIG. 9B. The temperature dependence of magnetization in intermediate fields (20 mT and 364 mT) revealed a more complicated behavior that is associated with a competition between Zeeman, dipolar, and anisotropy fields.

The ⁵⁷Fe Mössbauer absorption spectra was also measured as a function of temperature without external magnetic field. These spectra have a relaxation character due to oscillations of domain walls. A complete quench of the magnetic component was observed at 290 K, consistent with the SQUID measurements at 2 mT.

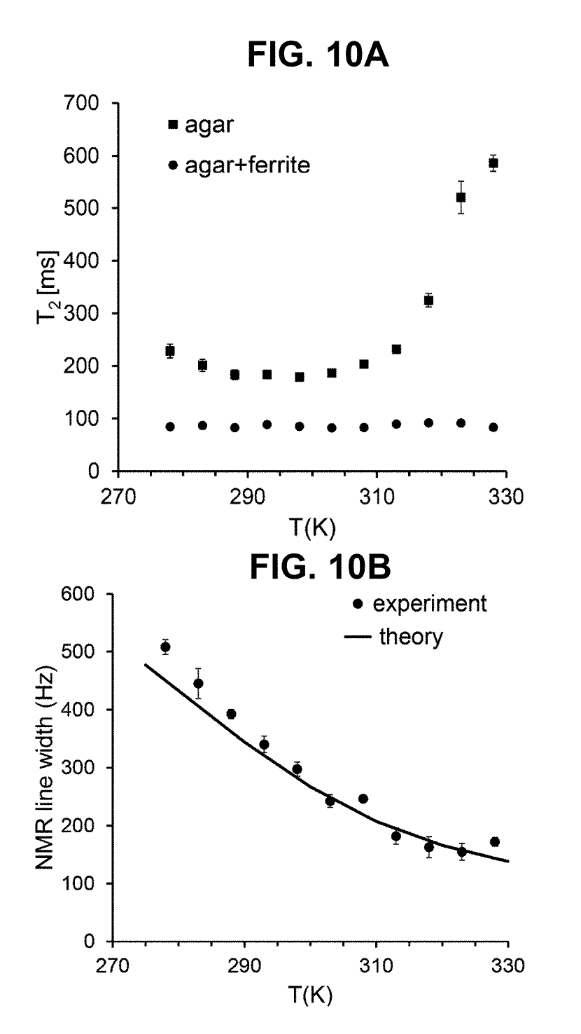

T₂ nuclear relaxation times as a function of temperature were measured using the Carr-Purcell-Meiboom-Gill method and are shown in FIG. 10A. Despite the sudden increase of T₂ above 320 K in the pure agar sample, the value of T₂ in the agar doped with ferrite particles remains constant through the entire measured temperature range. FIG. 10B shows the results of NMR linewidth broadening, i.e. the difference in linewidth for agar gel samples with and without the ferrite particles. The linewidth measurements of the sample with the ferrite particles indicate a significant decrease of linewidth with temperature, consistent with the SQUID measurements showing a reduction in magnetization. A simple theoretical calculation of the linewidth created by the inhomogeneous magnetic field of the particles is also shown in FIG. 10B (solid line). This calculation has no adjustable parameters and uses the measured magnetization as a function of temperature at 364 mT. The agreement between theory and experiment is excellent, confirming the hypothesis that the temperature-dependent linewidth comes from the inhomogeneous fields of the magnetic particles. The rapid decrease in linewidth with increasing temperature suggests a significant temperature-dependent change in brightness in the T₂* weighted MR imaging.

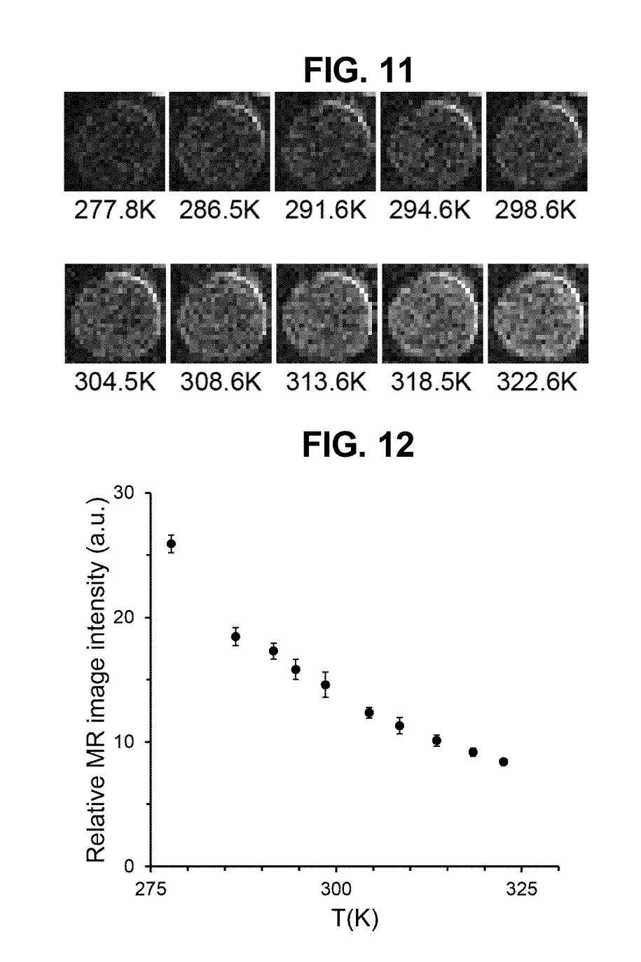

Gradient-echo, T₂* weighted, axial images of the agar gel doped with ferrite particles at different temperature are shown in FIG. 11. As anticipated from SQUID magnetization and NMR linewidth measurements, the intensity of the images increases significantly with the temperature increase. FIG. 12 shows a plot of the ratio of the intensity of the pure agar image to that of the agar sample doped with ferrite particles as a function of temperature. The image intensity was averaged across the entire area of the axial slice. It is clear from this data that the ferrite particles in this example, on average 2 microns in size, produced intensity changes necessary for temperature-dependent measurements.

The accuracy of temperature change determination from the phantom MR imaging was analyzed using linear regression in the range of 286 K to 323 K. From the regression's 95% confidence bands, a temperature resolution of 1.3 K at 305 K was obtained.

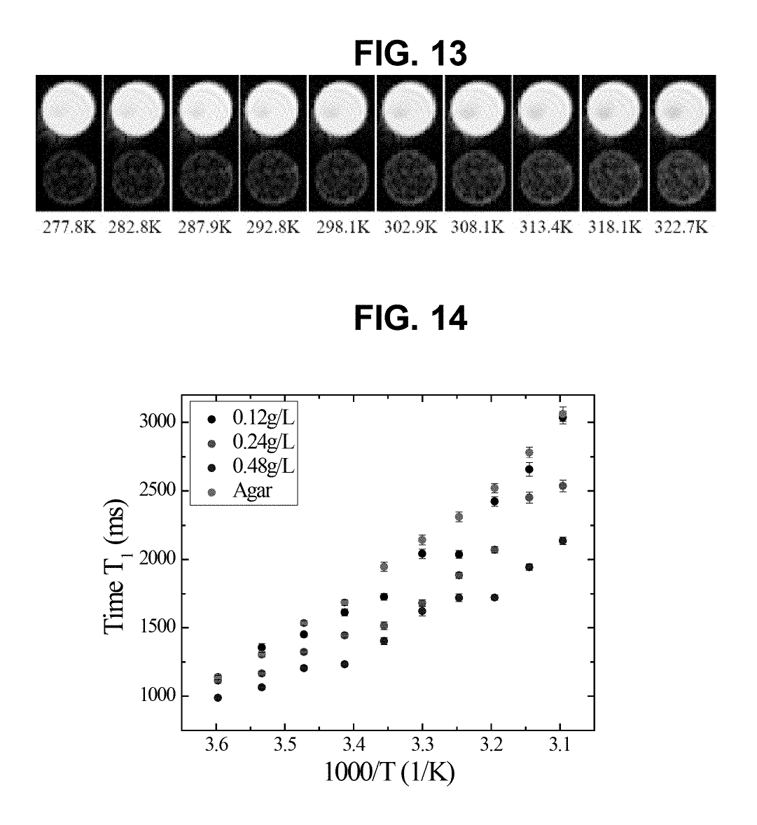

Similar results were obtained with Co_{0.3}Zn_{0.7}Fe₂O₄ nanoparticles. FIG. 13 shows a set of T₂* weighted, gradient-echo MR images of a 2% Ringer's solution-agarose sample with embedded Co_{0.3}Zn_{0.7}Fe₂O₄ nanoparticles at various temperatures. These results illustrate that ferrite nanoparticles of the present invention can be used as an MRI temperature contrast agent. In addition, FIG. 14 shows the temperature dependence of the T₁ relaxation time. This result indicates that T₁ relaxation times (in addition to the T₂* relaxation time) can be measured to obtain spatial temperature maps by MRI thermometry.

The foregoing examples of the present disclosure have been presented for purposes of illustration and description. Furthermore, these examples are not intended to limit the disclosure to the form disclosed herein. Consequently, variations and modifications commensurate with the teachings of the description of the disclosure, and the skill or knowledge of the relevant art, are within the scope of the present disclosure. The specific embodiments described in the examples provided herein are intended to further explain the best mode known for practicing the disclosure and to enable others skilled in the art to utilize the disclosure in such, or other, embodiments and with various modifications required by the particular applications or uses of the present disclosure. It is intended that the appended claims be construed to include alternative embodiments to the extent permitted by the prior art.

All references throughout this application (for example, patent documents including issued or granted patents or equivalents; patent application publications; and non-patent literature documents or other source material) are hereby incorporated by reference herein in their entireties, as though individually incorporated by reference, to the extent each reference is at least partially not inconsistent with the disclosure in this application (for example, a reference that is partially inconsistent is incorporated by reference except for the partially inconsistent portion of the reference).

Those skilled in the art will recognize, or be able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application. In general, the terms and phrases used herein have their art-recognized meaning, which can be found by reference to standard texts, journal references and contexts known to those skilled in the art. Any preceding definitions are provided to clarify their specific use in the context of the invention.

It is to be understood that wherever values and ranges are provided herein, all values and ranges encompassed by these values and ranges, are meant to be encompassed within the scope of the present invention. Moreover, all values that fall within these ranges, as well as the upper or lower limits of a range of values, are also contemplated by the present application.

The disclosures of all patents, patent applications, and publications referenced by this disclosure are incorporated herein by reference in their entireties. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. An MRI thermometry contrast agent, comprising a ferrite compound of the formula (M₁₋ₓYₓ)_{z}Fe_{3-z}O₄, wherein M and Y represent metallic elements, wherein the ferrite compound has a Curie transition temperature between about 150 K and about 350 K, and wherein a magnitude of a change in magnetization of the ferrite compound as a function of temperature, in a magnetic field of between about 0.2 teslas and about 7 teslas and a temperature range between about 278 K and about 333 K, is at least about 0.01 Am²·kg⁻¹·K⁻¹.

2. The MRI thermometry contrast agent of claim 1, wherein the ferrite compound is in the form of particles.

3. The MRI thermometry contrast agent of claim 2, wherein a mean particle size of the particles is between about 5 nm and about 5 μm.

4. A pharmaceutical composition suitable for administration to a human subject, comprising:
a ferrite compound of the formula (M₁₋ₓYₓ)_{z}Fe_{3-z}O₄, wherein M and Y represent metallic elements, wherein the ferrite compound has a Curie transition temperature between about 150 K and about 350 K, and wherein a magnitude of a change in magnetization of the ferrite compound as a function of temperature, in a magnetic field of between about 0.2 teslas and about 7 teslas and a temperature range between about 278 K and about 333 K, is at least about 0.01 Am²·kg⁻¹·K⁻¹; and
at least one pharmaceutically acceptable carrier or excipient.

5. The pharmaceutical composition of claim 4, wherein the composition is suitable for administration to a human subject by a route selected from the group consisting of inhalational, oral, nasal, rectal, parenteral, sublingual, transdermal, transmucosal, intravesical, intrapulmonary, intraduodenal, intragastrical, intrathecal, epidural, intrapleural, intraperitoneal, intratracheal, otic, intraocular, subcutaneous, intramuscular, intradermal, intra-arterial, intravenous, intrabronchial, and topical.

6. The pharmaceutical composition of claim 4, wherein a concentration of the ferrite compound in the composition is between about 0.04 mM and about 4 mM.

7. A method for non-invasively measuring a temperature of a portion of a body of a human subject, comprising:
administering to the subject a composition comprising a ferrite compound of the formula (M₁₋ₓYₓ)_{z}Fe_{3-z}O₄, wherein M and Y represent metallic elements, wherein the ferrite compound has a Curie transition temperature between about 150 K and about 350 K, and wherein a magnitude of a change in magnetization of the ferrite compound as a function of temperature, in a magnetic field of between about 0.2 teslas and about 7 teslas and a temperature range between about 278 K and about 333 K, is at least about 0.01 Am²·kg⁻¹·K⁻¹;
collecting at least one magnetic resonance image of the subject;
measuring at least one relaxation time of the composition in the subject, the at least one relaxation time selected from the group consisting of T₁ relaxation time, T₂ relaxation time, and T₂* relaxation time; and
determining the temperature of the portion of the body of the human subject using the at least one magnetic resonance image and the at least one relaxation time.

8. The method of claim 7, wherein a measurement error of the measured temperature of the human subject is no more than about 1° C.

9. The method of claim 7, wherein the composition is administered to the subject by a route selected from the group consisting of inhalational, oral, nasal, rectal, parenteral, sublingual, transdermal, transmucosal, intravesical, intrapulmonary, intraduodenal, intragastrical, intrathecal, epidural, intrapleural, intraperitoneal, intratracheal, otic, intraocular, subcutaneous, intramuscular, intradermal, intra-arterial, intravenous, intrabronchial, topical, injection, insertion into the body, and covering a portion of an external surface of the patient.

10. An article for use in MRI thermometry of a human subject, comprising:
a medium, comprising a nuclide with non-zero nuclear spin; and
a ferrite compound of the formula (M₁₋ₓYₓ)_{z}Fe_{3-z}O₄, wherein M and Y represent metallic elements, wherein the ferrite compound has a Curie transition temperature between about 150 K and about 350 K, and wherein a magnitude of a change in magnetization of the ferrite compound as a function of temperature, in a magnetic field of between about 0.2 teslas and about 7 teslas and a temperature range between about 278 K and about 333 K, is at least about 0.01 Am²·kg⁻¹·K⁻¹,
wherein a concentration of the ferrite compound in the article is between about 0.05 g/L and about 5 g/L.

11. The article of claim 9, wherein the article is selected from the group consisting of a phantom in the form of a container, a phantom in the form of a thick film, and an outer layer of a device, the device selected from the group consisting of a catheter, an MRI-guided treatment tool, an implant, a probe, an applicator, a mesh, and a stent.

12. The article of claim 10, wherein the article is a phantom in the form of a container, wherein the container is suitable for placement in a body cavity of the human subject and is selected from the group consisting of an ampoule and a film.

13. The article of claim 10, wherein the article is a phantom in the form of a thick film, wherein the thick film is configured to cover skin of the human subject and allow MRI thermometry of the skin during an MRI-guided medical procedure.

14. A method for non-invasively measuring an internal temperature of an object, the object comprising a nuclide with non-zero nuclear spin, the method comprising:
placing, in an interior of the object, a composition comprising a ferrite compound of the formula (M₁₋ₓYₓ)_{z}Fe_{3-z}O₄, wherein M and Y represent metallic elements, wherein the ferrite compound has a Curie transition temperature between about 150 K and about 350 K, and wherein a magnitude of a change in magnetization of the ferrite compound as a function of temperature, in a magnetic field of between about 0.2 teslas and about 7 teslas and a temperature range between about 278 K and about 333 K, is at least about 0.01 Am²·kg⁻¹·K⁻¹;
collecting at least one magnetic resonance image of the object;
measuring at least one relaxation time of the composition in the object, the at least one relaxation time selected from the group consisting of T₁ relaxation time, T₂ relaxation time, and T₂* relaxation time; and
determining the temperature of the object using the at least one magnetic resonance image and the at least one relaxation time.

15. A method for measuring a temperature within a tissue of a human subject, comprising:
providing a contrast agent, wherein the contrast agent has a Curie transition temperature between about 150 K and about 350 K, and wherein a magnitude of a change in magnetization of the contrast agent as a function of temperature, in a magnetic field of between about 0.2 teslas and about 7 teslas and a temperature range between about 278 K and about 333 K, is at least about 0.01 Am²·kg⁻¹·K⁻¹;
deploying the contrast agent into the tissue;
subjecting the tissue to magnetic resonance imaging (MRI); and
measuring at least one quantity selected from the group consisting of 1) linewidth broadening by image-guided localized nuclear magnetic resonance spectroscopy and 2) a ratio of MRI image intensity between MRI images of the tissue before and after deployment of the contrast agent; and
associating the measured quantity with the temperature within the tissue.

16. The method of claim 15, wherein the contrast agent comprises gadolinium particles comprising at least one dopant selected from the group consisting of cobalt and copper.

17. The method of claim 15, wherein the tissue further comprises a medical implant.

18. The method of claim 17, wherein the medical implant is a metal-containing implant.

19. The method of claim 15, wherein the contrast agent is deployed within a phantom.

20. The method of claim 19, wherein the phantom comprises at least one selected from the group consisting of a polymeric film, a catheter, an MRI-guided treatment tool, an implant, a probe, an applicator, a mesh, an ampoule, a film, and a stent.
